(19) United States
(12) Patent Application Publication
(10) Pub. No.: US 2025/0243145 A1
(43) Pub. Date: Jul. 31, 2025

(54) Supercritical Water Oxidation to Treat Biomass and Organic Waste to Produce Chemical Products and Sodium Formate

(71) Applicant: NuScale Power, LLC, Corvallis, OR (US)

(72) Inventors: Francis Y. Tsang, Bellingham, WA (US); José N. Reyes, JR., Corvallis, OR (US); Luis DePavia, Corvallis, OR (US)

(21) Appl. No.: 19/037,946

(22) Filed: Jan. 27, 2025

Related U.S. Application Data
(60) Provisional application No. 63/626,446, filed on Jan. 29, 2024.

(51) Int. Cl. C07C 51/10; C01B 3/02; C01B 7/01

(52) U.S. Cl. CPC C07C 51/10; C01B 3/02; C01B 7/012

(57) ABSTRACT

An integrated energy system comprising a power plant including at least one nuclear reactor and an electrical power generation system, the at least one nuclear reactor being configured to generate steam, and a supercritical water oxidation system operably coupled to the power plant. The supercritical water oxidation system including a desalination plant configured to produce first water and brine, a chlor-alkali membrane process configured to receive the brine and produce at least a Sodium Hydroxide solution, a reactor configured to receive the first water, the steam, and the Sodium Hydroxide solution to produce a waste solution and a solid waste, and a separator configured to receive the waste solution and produce Carbon Dioxide and second water.

### CROSS REFERENCE TO RELATED APPLICATIONS

This application claims the benefit of U.S. Provisional Patent Application No. 63/626,446, filed Jan. 29, 2024, and titled “SUPERCRITICAL WATER OXIDATION TO TREAT BIOMASS AND ORGANIC WASTE TO PRODUCE CHEMICAL PRODUCTS AND SODIUM FORMATE,” which is incorporated herein by reference in its entirety.

### BACKGROUND

The growing public concern of hazardous waste disposal began with the Resource Conservation and Recovery Act (RCRA) in 1976. Although recycling became an increasingly popular way to handle hazardous waste, it is not always viable and/or practical. Many industrial hazardous wastes, such as complex organic material and sludges, organic and inorganic compounds, halogenated and sulfur-bearing organic and inorganic substances, as well as military energetic materials are extremely difficult to treat and dispose. Currently, there are to acceptable industrial practices to treat hazardous waste.

The first industrial practice is chemical treatment, such as neutralization, precipitation, ion exchange, reduction, or oxidation. The second industrial practice is thermal treatment, such as incineration. But both practices generate unnecessary by-products and carcinogen chemicals if released into the environment. Because the existence practices cause harm to the environment, improvement is necessary.

### BRIEF DESCRIPTION OF THE DRAWINGS

FIG. 1 schematically illustrates a representation of an integrated energy system that includes a small modular reactor (SMR) system integrated with a chemical production process utilizing supercritical water oxidation (SCWO) technology, according to an embodiment of this disclosure.

FIG. 2 illustrates a flow diagram of an example process associated with Hydrochloric Acid (HCl) production and Carbon Dioxide (CO₂) production using SCWO, according to an embodiment of this disclosure.

FIG. 3 illustrates a flow diagram of an example process associated with using Carbon Dioxide (CO₂) produced by a separator to generate Oxygen (O₂), Carbon Monoxide (CO), and Carbon Dioxide (CO₂) using a solid oxide electrolyzer cell (SOEC) stack (e.g., CO-Electrolysis Cell, etc.) according to an embodiment of this disclosure.

FIG. 4 illustrates a block diagram of a chlor-alkali membrane process, according to an embodiment of this disclosure.

FIG. 5 illustrates a flow diagram of an example system associated with using a power plant system (e.g., SMR system) and a SCWO-R to produce Sodium Formate (HCOONa), according to an embodiment of this disclosure.

FIG. 6 schematically illustrates an example process for the generation of Sodium Formate (HCOONa) from Sodium Hydroxide (NaOH) and Carbon Monoxide (CO), and the thermal decomposition of the Sodium Formate (HCOONa) to Sodium Oxalate ((COO)₂Na₂)) and Hydrogen (H₂), according to an embodiment of this disclosure.

FIG. 7 is a partially schematic, partially cross-sectional view of a nuclear reactor system configured in accordance with embodiments of the present technology, according to an embodiment of this disclosure.

FIG. 8 is a partial schematic, partial cross-sectional view of a nuclear reactor system configured in accordance with additional embodiments of the present technology, according to an embodiment of this disclosure.

FIG. 9 is a schematic view of a nuclear power plant system including multiple nuclear reactors in accordance with embodiments of the present technology, according to an embodiment of this disclosure.

FIG. 10 illustrates a flow diagram of an example process for utilizing a small modular nuclear reactor (SMR) system to produce Sodium Formate (HCOONa), according to an embodiment of this disclosure.

FIG. 11 illustrates a flow diagram of an example process for utilizing a small modular nuclear reactor (SMR) system and supercritical water oxidation to treat biomass and/or organic waste, according to an embodiment of this disclosure.

### DETAILED DESCRIPTION

### Overview

The terminology used in the Detailed Description is intended to be interpreted in its broadest reasonable manner, even though it is being used in conjunction with a detailed description of certain specific embodiments of the disclosure.

Certain terms may even be emphasized below; however, any terminology intended to be interpreted in any restricted manner will be overtly and specifically defined as such in this Detailed Description section.

The headings provided herein are for convenience only and should not be construed as limiting the subject matter disclosed.

Supercritical water oxidation (SCWO) is a high-efficiency, thermal oxidation process that may be capable of treating a wide variety of hazardous wastes at elevated temperatures and pressures; exceeding the thermodynamic critical point of water, P=220 bar, T>375° C., SCWO is ideally suited for treating waste streams containing high concentration of water.

This process may transform biomass and organic matter into water, carbon dioxide, and depending on the waste undergoing treatment, an inert mineral solid residue.

The Detailed Description is set forth with reference to the accompanying figures. In the figures, the left-most digit(s) of a reference number identifies the figure in which the reference number first appears. The use of the same reference numbers in different figures indicates similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

This disclosure is directed to the use of supercritical water oxidation (SCWO) technology to convert biomass and/or organic matter into an inert mineral waste and useful chemical components that may be used for chemical production. SCWO is a single step wet oxidation process that transform and/or decomposes biomass and organic water into mainly water and Carbon Dioxide (CO₂), and, in some cases, an inert mineral solid residue. Some key characteristics of supercritical water are its viscosity and dielectric constants.

Supercritical water (SCW) exists at a temperature of approximately 374° C. (647 K) and at a pressure of approximately 22.1 MPa (218 atm), which is considered its critical point. The viscosity of SCW is an order of magnitude smaller than its liquid phase (i.e., ordinary water), which makes it easier to diffuse between solute molecules compared to ordinary water.

The dielectric constant of SCW is more than an order of magnitude lower than that of ordinary water, that may make it an effective solvent to break the chemical bonds in most biomass and organic matter. Ordinary water may dissolve most inorganic substances, but most organic substances and gases exhibit very low solubility. SWO also shows almost opposite properties (i.e., almost insoluble to inorganic matter, but soluble to organic matter and gas). SWO may be a very useful reaction medium because of its unusual solubility in organic matter and gas. Under supercritical conditions, organic compounds and an oxidation agent (e.g., Oxygen (O₂), Hydrogen Peroxide (H₂O₂), etc.), may become fully miscible in water, allowing oxidation to occur in a single fluid phase with excellent transport properties. Many organic compounds are completely oxidized in under one minute with optimized temperature. Processing wastewater and sewage has been a common application for SCWO.

SCWO technology may allow for the complete destruction of biomass and persistent toxic organic contaminants. As an alternative to using an oxidation agent (e.g., Oxygen (O₂), Hydrogen Peroxide (H₂O₂), etc.) as an oxidant in SCWO, Sodium Hydroxide (NaOH), which can be generated from brine treatment after it is discharged from a seawater desalination plant (e.g., reverse osmosis, flash-distilling type, etc.) may be used to neutralize acidic byproducts. In an embodiment, Sodium Hydroxide (NaOH) may be used to neutralize acids, adjust pH levels, and act as a nucleophile in certain organic reactions (e.g., dissolve metals from solutions by precipitating them as solid hydroxides, etc.). The produced Carbon Dioxide (CO₂) and water may be used to generate valuable chemical products such as Methanol (CH₃OH), Formaldehyde (CH₂O), Acetic Acid (CH₃COOH), and synthetic fuels as well as Sodium Formate (HCOONa), which is a Hydrogen energy carrier. Also, the SCWO processing systems may be fully enclosed and may not produce hazardous air pollutants (HAPS) or Nitrogen-carrying pollutants (e.g., Nitric Oxide (NO), Nitrogen Dioxide (NO₂), etc.).

The large degree of variability between biomass resources, both currently available and emerging, may be a significant barrier to the utilization of biomass as a feedstock for fuel and chemical production. The impacts of physical characteristics (e.g., moisture content, particle size, etc.) and chemical characteristics (e.g., ash content, extractives/volatiles, lignin, etc.) play varying, and intricate, roles during biomass conversion. Currently, there are several known thermal decomposition technologies to treat biomass, but all require a long decomposition time (reaction time) and produce contaminated water and other harmful by-products (e.g., nitrogen oxides (NOx), Sulphur Oxides (SOx), and/or Carbon Monoxide (CO)). Also, these known thermal decomposition processes are endothermic and require large quantities of energy for the entire treatment system. These known thermal decomposition processes require uninterrupted and continuous energy supply systems, and renewable energy sources are not applicable for these specific applications.

SCW is a dense single-phase fluid with transport properties similar to those of a gas, and solvent properties comparable to those of a non-polar solvent. The oxidation agent (e.g., Oxygen (O₂), Hydrogen Peroxide (H₂O₂), etc.), may be fully soluble in SWO, which results in the rapid and complete oxidation of all organic material to Carbon Dioxide (CO₂), clean water (H₂O), and some non-leachable inorganic salts. SWO may be a very useful reaction medium because of its unusual solubility in organic matter and gas.

There are many advantages to SCWO treatment. SCWO is a destructive treatment in that the compounds being treated are mineralized to simple elements (e.g., water and Carbon Dioxide (CO₂)) rather than just being transferred to another medium. SCWO may be a complete oxidation process and reactions of by-products via oxidized contaminants may be eliminated. SCWO may be extremely rapid. For example, typical reaction times may occur in approximately 5-10 seconds, making it possible to have systems that may be very compact and have a high throughput. The highly oxidizing environment may make it possible to effectively treat all sorts of organic contaminants with very high (i.e., >99%) destruction efficiencies. This may include treatment of trace contaminants, slurries of biosolids, waste oil, food wastes, plastics, and/or emerging contaminants such as PFAS or 1,4-dioxane.

The relatively moderate temperatures of SCWO process (i.e., 400-600° C.) as compared to other destructive technologies, such as incineration, may prevent the formation of Nitrogen-carrying compounds (i.e., NO, NO₂, NOₓ, etc.), Sulfur-carrying compounds (i.e., SO, SO₂, SOₓ, etc.), and dioxins. Also, SCWO treatment does not require drying of the waste before treatment, so both liquids and slurries may be treated using SCWO.

An SMR (e.g., NuScale VOYGR NPP) may provide uninterrupted power, thermal, and process steam to support the SCWO process. SCWO may use Oxygen (O₂) or Hydrogen Peroxide (H₂O₂) as an oxidant to break down organic substances in water. A Sodium Hydroxide (NaOH) solution may be added to the SCWO process as a catalyst to accelerate and to enhance the oxidation reactions. Generating high-quality water from seawater using a desalination process (e.g., reverse osmosis, flash-type distilling, etc.) may produce brine (i.e., a concentrated NaCl solution). Brine may be treated via a Chlor-Alkali-Membrane process to generate NaOH.

### Illustrative Embodiments

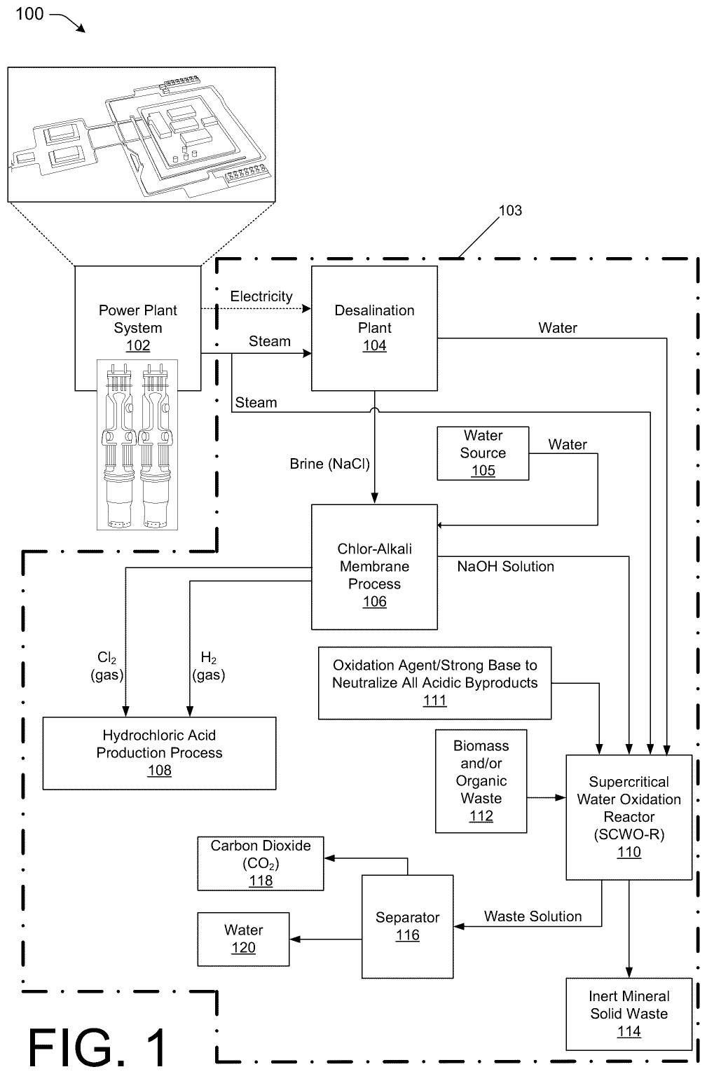

Specifically, FIG. 1 schematically illustrates a representation of an integrated energy system 100 (“system 100”) that includes a power plant system 102 (e.g., a small modular reactor (SMR) system) integrated with a chemical production process utilizing supercritical water oxidation (SCWO) technology, according to an embodiment of this disclosure.

In an embodiment, the system 100 may include the power plant system 102 and a supercritical water oxidation (SCWO) system 103. In an embodiment, the SCWO system 103 may include a desalination plant 104, a water source 105, a chlor-alkali membrane process 106, a Hydrochloric Acid (HCl) production process 108, a supercritical water oxidation reactor (SCWO-R) 110, an oxidation agent/strong base to neutralize all acidic byproducts 111, biomass and/or organic waste 112, inert mineral solid waste 114, a separator 116, Carbon Dioxide (CO₂) 118, and water 120. In an embodiment, the oxidation agent/strong base to neutralize all acidic byproducts 111 may include Oxygen (O₂), Hydrogen Peroxide (H₂O₂), or Sodium Hydroxide (NaOH), etc.

In an embodiment, the power plant system 102 may be configured for use in one or more industrial processes/operations and, more particularly, for use in resource production and SCWO operations. The power plant system 102 may be located at or near the location of the SCWO-R 110. For example, the power plant system 102 may be a permanent or temporary installation built at or near (e.g., roughly 1 km) the location of the SCWO-R 110 or may be a mobile or partially mobile system that is moved to and assembled at or near (e.g., within a threshold distance from) the location of the SCWO-R 110. For example, the power plant system 102 may be moved to and assembled at or near any other portions of the system 100.

In an embodiment, the power plant system 102 may include an SMR system (e.g., multi-module power plant design). However, in various instances, the power plant system 102 may represent any type of power plant system including any of various other types of nuclear reactors and/or nuclear reactor systems.

The power plant system 102 may be operably coupled to the desalination plant 104, the chlor-alkali membrane process 106, the Hydrochloric Acid (HCl) production process 108, the SCWO-R 110, the separator 116, and/or additional components for resource production and/or SCWO operations. The power plant system 102 may be referred to as a primary subsystem for carrying out the resource production and/or SCWO operations. The desalination plant 104, the chlor-alkali membrane process 106, the Hydrochloric Acid (HCl) production process 108, the SCWO-R 110, and the separator 116 may be referred to as a secondary subsystem for carrying out a secondary process.

In an embodiment, the power plant system 102 may be electrically coupled to the to the desalination plant 104, the chlor-alkali membrane process 106, the Hydrochloric Acid (HCl) production process 108, the supercritical water oxidation reactor (SCWO-R) 110, the separator 116, and/or additional components for resource production and/or SCWO operations for selectively providing electricity (e.g., power) thereto. Similarly, individual ones of steam output paths of the power plant system 102 may be fluidly coupled to the desalination plant 104 and/or the SCWO-R 110 for selectively providing steam thereto. In an embodiment, the power plant system 102 may be operably coupled to additional or fewer outputs and/or the various outputs can receive electricity and/or steam from other sources (e.g., conventional steam suppliers, conventional electricity sources, etc.).

It is noted that the desalination plant 104 may be used for the desalination of seawater or any other brackish water sources via reverse osmosis distillation, flash-type boiling desalination, or any other available desalination process. The desalination plant 104 may receive power (e.g., electricity) from the power plant system 102 via one or more electrical output paths from an electrical power transmission system (not shown) of the power plant system 102. In an embodiment, the desalination plant 104 may perform water desalination using steam received from the power plant system 102 via a steam supplier (e.g., heat exchanger, steam generator, steam connection, etc.) from a steam transmission system (not shown) of the power plant system 102.

The chlor-alkali membrane process 106 may be configured to receive brine from the desalination plant 104 and clean water from a water source 105. In an embodiment, the chlor-alkali membrane process 106 may process the brine to generate Chlorine (Cl₂) gas, Hydrogen (H₂) gas, and a Sodium Hydroxide (NaOH) solution.

In an embodiment, the water source 105 may be the desalination plant 104. In an embodiment, the chlor-alkali membrane process 106 may be configured to remove impurities from the brine received from the desalination plant 104. For example, the brine may undergo precipitation and filtration to remove impurities.

In an embodiment, the Hydrochloric Acid (HCl) production process 108 may receive the Chlorine (Cl₂) gas and Hydrogen (H₂) gas to produce Hydrochloric Acid (HCl) as demonstrated by Equation 1:

$${Cl}_{2} + {H}_{2} = 2 HCl \qquad ( 1 )$$

In an embodiment, the SCWO-R 110 may be configured to receive the oxidation agent/strong base to neutralize all acidic byproducts 111 (e.g., Oxygen (O₂), Hydrogen Peroxide (H₂O₂), Sodium Hydroxide (NaOH), etc.), biomass and/or organic waste 112, high-quality water from the desalination plant 104, steam from the power plant system 102, and a Sodium Hydroxide (NaOH) solution from the chlor-alkali membrane process 106. In an embodiment, the biomass and/or organic waste 112 may be wet or dry. For example, the biomass and/or organic waste 112 may include slurries of biosolids, waste oil, food wastes, plastics, and/or emerging contaminants such as PFAS or 1,4-dioxane.

In an embodiment, the oxidation agent/strong base to neutralize all acidic byproducts 111 may include a Sodium Hydroxide (NaOH) solution generated by the chlor-alkali membrane process 106.

In an embodiment, the SCWO-R 110 may utilize the steam from the power plant system 102 to increase the pressure and temperature within the SCWO-R 110 to the critical temperature and pressure necessary to generate SCW from the water supplied by the desalination plant 104. The SCWO-R 110 may utilize the Sodium Hydroxide (NaOH) solution as a catalyst to encourage SCWO and to increase the rate at which the SCWO-R 110 may decompose/convert the biomass and/or organic waste 112. In an embodiment, the SCWO-R 110 may be configured to decompose/convert the biomass and/or organic waste 112 into inert mineral solid waste 114, and a waste solution. The waste solution may be directed into the separator 116. In an embodiment, the separator 116 may be configured to separate the waste solution into Carbon Dioxide (CO₂) 118 and water 120.

For clarity, a certain number of components are shown in FIG. 1. It is understood, however, that an embodiment of this disclosure may include more than one of each component. In addition, an embodiment of this disclosure may include fewer than or greater than all of the components shown in FIG. 1. In addition, the components in FIG. 1 may communicate via any suitable communication medium (including the Internet), using any suitable communication protocol.

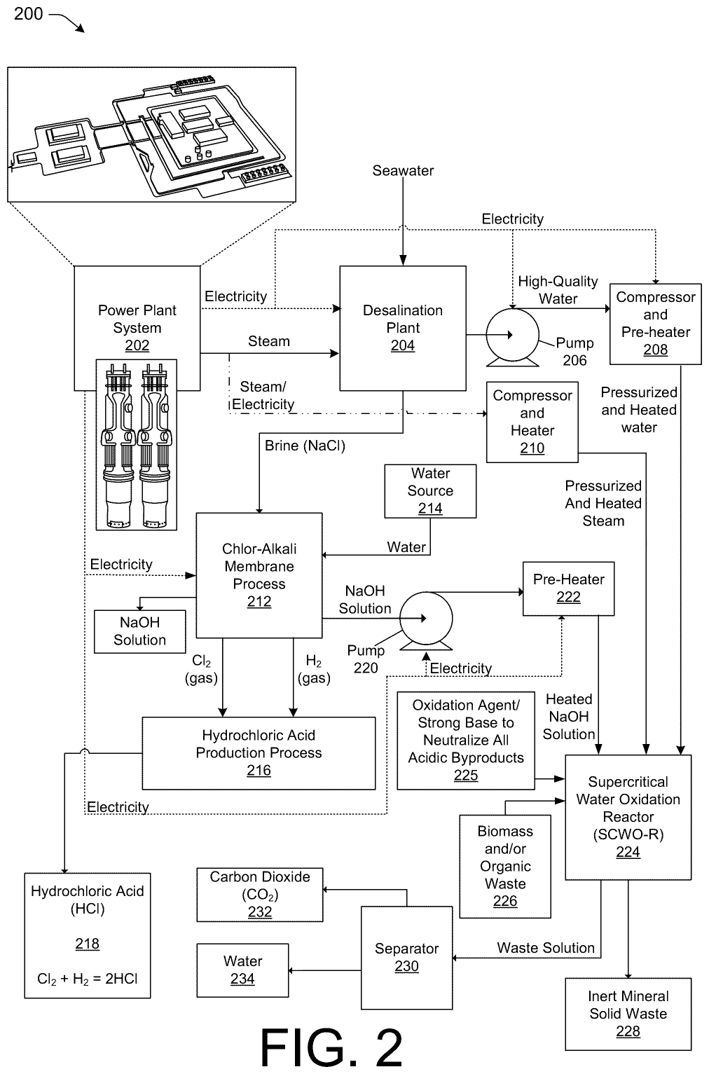

FIG. 2 illustrates a flow diagram of an example system 200 (“system 200”) associated with the Hydrochloric Acid (HCl) production process 118 and the production of the Carbon Dioxide (CO₂) 118 of FIG. 1 using SCWO, according to an embodiment of this disclosure. The order in which the operations or steps are described is not intended to be construed as a limitation, and any number of the described operations or steps may be combined in any order and/or in parallel during the operation of system 200. The system 200 depicted in FIG. 2 may be the same or similar to the system 100 depicted in FIG. 1. For example, in an embodiment, the system 200 depicted in FIG. 2 may be utilized to provide power and steam to the desalination plant 104, the chlor-alkali membrane process 106, and the SCWO-R 110, as depicted in FIG. 1. Similarly, in an embodiment, the power plant system 102 depicted in FIG. 1 may be utilized to provide steam and electricity to the desalination plant 204, the chlor-alkali membrane process 212, and the SCWO-R 224, as depicted in FIG. 2.

In an embodiment, the system 200 may include a power plant system 202, a desalination plant 204, a pump 206, a compressor and pre-heater 208, compressor and heater 210, a chlor-alkali membrane process 212, a water source 214, a hydrochloric acid production process 216, a hydrochloric acid 218, a pump 220, a pre-heater 222, a supercritical water oxidation reactor (SCWO-R) 224, an oxidation agent/strong base to neutralize all acidic byproducts 225, biomass and/or organic waste 226, inert mineral solid waste 228, a separator 230, Carbon Dioxide (CO₂) 232, and water 234. In an embodiment, the oxidation agent/strong base to neutralize all acidic byproducts 225 may include an Oxygen (O₂), Hydrogen Peroxide (H₂O₂), or Sodium Hydroxide (NaOH).

In an embodiment, the power plant system 202 may be configured for use in one or more industrial processes/operations and, more particularly, for use in resource production (e.g., hydrochloric acid production, Carbon Dioxide (CO₂) production, etc.) and SCWO operations. The power plant system 202 may be located at or near the location of the SCWO-R 224. For example, the power plant system 202 may be a permanent or temporary installation built at or near (e.g., roughly 1 km) the location of the SCWO-R 224 or may be a mobile or partially mobile system that is moved to and assembled at or near (e.g., within a threshold distance from) the location of the SCWO-R 224. For example, the power plant system 202 may be moved to and assembled at or near any other portions of the system 200.

In an embodiment, the power plant system 202 may include an SMR system (e.g., multi-module power plant design). However, in various instances, the power plant system 202 may represent any type of power plant system including any of various other types of nuclear reactors and/or nuclear reactor systems.

The power plant system 202 may be operably coupled to the desalination plant 204, the chlor-alkali membrane process 212, the Hydrochloric Acid (HCl) production process 216, the SCWO-R 224, the separator 230, and/or additional components for resource production and/or SCWO operations. The power plant system 202 may be referred to as a primary subsystem for carrying out the resource production and/or SCWO operations. The desalination plant 204, the chlor-alkali membrane process 212, the Hydrochloric Acid (HCl) production process 216, the SCWO-R 224, and the separator 230 may be referred to as a secondary subsystem for carrying out a secondary process.

In an embodiment, the power plant system 202 may be electrically coupled to the to the desalination plant 204, the chlor-alkali membrane process 212, the Hydrochloric Acid (HCl) production process 216, the SCWO-R 224, the separator 230, and/or additional components for resource production and/or SCWO operations for selectively providing electricity (e.g., power) thereto. Similarly, individual ones of steam output paths of the power plant system 202 may be fluidly coupled to the desalination plant 204 and/or the SCWO-R 224 for selectively providing steam thereto. In an embodiment, the power plant system 202 may be operably coupled to additional or fewer outputs and/or the various outputs can receive electricity and/or steam from other sources (e.g., conventional steam suppliers, conventional electricity sources, etc.).

In an embodiment, the desalination plant 204 may be used for the desalination of seawater or any other brackish water source via reverse osmosis distillation, flash-type boiling desalination, or any other available desalination process. The desalination plant 204 may receive power (e.g., electricity) from the power plant system 202 via one or more electrical output paths from an electrical power transmission system (not shown) of the power plant system 102. In an embodiment, the desalination plant 204 may perform water desalination using steam received from the power plant system 202 via a steam supplier (e.g., heat exchanger, steam generator, steam connection, etc.) from a steam transmission system (not shown) of the power plant system 202.

In an embodiment, the desalination plant 204 may be configured to receive seawater. The desalination plant 204 may utilize the steam and the electricity from the power plant system 202 to produce high quality water and a brine (i.e., a concentrated Sodium Chloride (NaCl) solution). The high quality water may be used for downstream processing. The brine may be directed to the chlor-alkali membrane process 212 for processing.

In an embodiment the pump 206 may transport the high quality water produced by the desalination plant 204 from the desalination plant 204 to the compressor and pre-heater 208. In an embodiment, the compressor and pre-heater 208 may receive electricity from the power plant system 202 and the high quality water discharged from the pump 206. In an embodiment, the compressor and pre-heater 208 may pressurize and heat the high-quality water to a threshold pressure and threshold temperature (e.g., parameters required to generate supercritical water, or any other desired parameters) to generate pressurized and heated water. The pressurized and heated water may be directed to the SCWO-R 224.

In an embodiment, the compressor and heater 210 may be configured to receive electricity and steam from the power plant system 202. The compressor and heater 210 may be configured to utilize the electricity from the power plant system 202 to pressurize and heat the steam from the power plant system 202 to a threshold pressure and threshold temperature to generate pressurized and heated steam. The pressurized and heated steam may be directed to the SCWO-R 224.

In an embodiment, the brine generated by the desalination plant 204 may be directed to the chlor-alkali membrane process 212 for processing. In an embodiment, the chlor-alkali membrane process 212 may be configured to receive electricity from the power plant system 202, brine from the desalination plant 204, and water from the water source 214. In an embodiment, the water source 214 may be the desalination plant 204. For example, the desalination plant 204 may discharge brine and high-quality water to the chlor-alkali membrane process 212. The chlor-alkali membrane process 212 may be configured to use the electricity from the power plant system 202 to convert the brine (NaCl solution) and the water (H₂O) to a Sodium Hydroxide (NaOH) solution, Chlorine (Cl₂) gas, and Hydrogen (H₂) gas. In an embodiment, the Chlorine (Cl₂) gas and the Hydrogen (H₂) gas may be directed to the Hydrochloric Acid Production Process 216. In an embodiment, the chlor-alkali membrane process 212 may produce an NaOH solution. In an embodiment, the chlor-alkali membrane process 216 may produce multiple Sodium Hydroxide (NaOH) solutions, each having a different concentrations.

In an embodiment, the NaOH solution may have a first specific concentration. In an embodiment, the NaOH solution with a first specific concentration may be discharged from the chlor-alkali membrane process 212. In an embodiment, pump 220 may transport another NaOH solution with a second specific concentration of from the chlor-alkali membrane process 212 to the pre-heater 222. In an embodiment, the pre-heater 222 may be configured to receive electricity from the power plant system 202. The pre-heater 222 may be configured to use the electricity from the power plant system 202 to heat the NaOH solution to the critical point to generate a heated NaOH solution. The heated NaOH solution may be directed to the SCWO-R 224.

In an embodiment, the oxidation agent/strong base to neutralize all acidic byproducts 225 may include Oxygen (O₂), Hydrogen Peroxide (H₂O₂), etc.), or Sodium Hydroxide (NaOH). In an embodiment, the oxidation agent/strong base to neutralize all acidic byproducts 225 may include Oxygen (O₂), Hydrogen Peroxide (H₂O₂), Sodium Hydroxide (NaOH), etc.). In an embodiment, the Sodium Hydroxide (NaOH) solution produced by the chlor-alkali membrane process 212 may be used as the oxidation agent/strong base to neutralize all acidic byproducts 225.

In an embodiment, the SCWO-R 224 may utilize the steam from the power plant system 202 to increase the pressure and temperature within the SCWO-R 224 to a threshold temperature and a threshold pressure necessary to generate SCW from the pressurized and heater water from the compressor and pre-heater 208. The SCWO-R 224 may utilize the heated Sodium Hydroxide (NaOH) solution from the pre-heater 222 as a catalyst to accelerate SCWO and to increase the rate at which the SCWO-R 224 may decompose/convert the biomass and/or organic waste 226. SCWO occurs at temperatures and pressures above water's critical point, which is 374.3° C. and 22.12 MPa. SCWO's unique conditions allow oxidation reactions to happen quickly, with over 99% completion in just a few minutes. The SCWO process produces Carbon Dioxide (CO₂), Nitrogen (N₂), Water (H₂O), mineral acids, inorganic salts, oxidized ash, and heat.

In an embodiment, the SCWO-R 224 may be configured to decompose/convert the biomass and/or organic waste 226 into inert mineral solid waste 228, and a waste solution. The waste solution may be directed into the separator 230. In an embodiment, the separator 230 may be configured to separate the waste solution into Carbon Dioxide (CO₂) 232 and water 234.

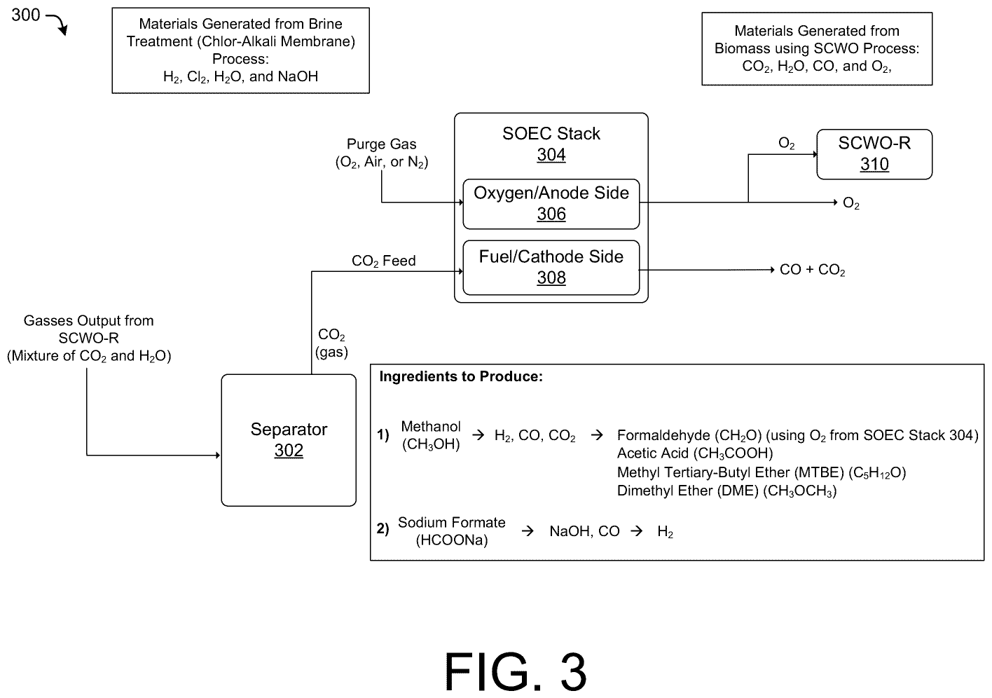

FIG. 3 illustrates a flow diagram of process 300 associated with using Carbon Dioxide (CO₂) produced by a separator 302 to generate Oxygen (O₂), Carbon Monoxide (CO), and Carbon Dioxide (CO₂) using a solid oxide electrolysis cell (SOEC) 304 (e.g., CO-Electrolysis Cell, etc.), according to an embodiment of this disclosure. Process 300 shows the continuous production of Oxygen (O₂), Carbon Monoxide (CO) and Carbon Dioxide (CO₂) from the Carbon Dioxide (CO₂) exhaust from the SCWO process. In an embodiment, the Oxygen produced by the SOEC stack 304 may be directed to the SCWO-R 310 for use as an oxidation agent (e.g., oxidation agent/strong base to neutralize all acidic byproducts 111, oxidation agent/strong base to neutralize all acidic byproducts 225).

In an embodiment, the process 300 may include the separator 302 and the SOEC stack 304. In an embodiment, the SOEC stack 304 may include an Oxygen/Anode side 306 and a Fuel/Cathode side 308. In an embodiment, the Carbon Dioxide (CO₂) discharged from the separator 302 may be fed into the fuel/cathode side 308 of the SOEC stack 304. Oxygen (O₂) from the reaction may be transported from the fuel/cathode side 308 to the Oxygen/Anode side 306 of the SOEC stack 304. The equation that governs the Carbon Dioxide (CO₂) reduction for Carbon Monoxide (CO) and Oxygen (O₂) production is shown in Equation 2:

2CO₂→CO₂+CO+½O₂ (2)

In an embodiment, air or Nitrogen (N₂) may be used to flush the Oxygen/Anode side 306 of the SOEC stack 304. In an embodiment, Carbon Dioxide (CO₂) may be used to flush the Oxygen/Anode side 306 of the SOEC stack 304, instead of air, to mitigate the risk of leakage of undesired gases, such as Nitrogen (N₂), into the Fuel/Cathode side 308 of the SOEC stack 304. Flushing the Oxygen/Anode side 306 of the SOEC stack 304 with Carbon Dioxide (CO₂) gas has two advantages: (1) enhancing the Oxygen (O₂) production concentration and (2) providing means for feeding energy into the SOEC stack 304.

In an embodiment, the SOEC stack 304 may be operated at elevated temperatures (e.g., ˜600° C.). In an embodiment, inlet gas to the Fuel/Cathode side 308 and/or flush gas to the Oxygen/Anode side 306 may be heated. For example, inlet gas may be heated in one or more auxiliary heaters (not shown), prior to entering the SOEC stack 304. In an embodiment, Joule heat (i.e., the heat produced when current is passed through the SOEC stack 304) may supply some or all of the heat necessary for the SOEC stack 304. In an embodiment, Joule heat, auxiliary heaters, and/or a means of heating known in the art may be used in combination to provide optimum operating conditions for the SOEC stack 304. In an embodiment, the process 300 may receive power and/or thermal energy from the power plant system 202.

The product stream from the Fuel/Cathode side 308 of the SOEC stack 304 may include Carbon Monoxide (CO) mixed with Carbon Dioxide (CO₂). The Carbon Monoxide (CO) and Carbon Dioxide (CO₂) mixture may be directed downstream for additional processing (e.g., pressure swing adsorption, storage, transportation, etc.).

In an embodiment, the Carbon Monoxide (CO) and Carbon Dioxide (CO₂) mixture may be combined with the Hydrogen (H₂) from a chlor-alkali membrane process (e.g., chlor-alkali membrane process 106, chlor-alkali membrane process 212) to produce Methanol (CH₃OH), Formaldehyde (CH₂O), Acetic Acid (CH₃COOH) and subsequent synfuels. In an embodiment, Sodium Formate (HCOONa), which is a Hydrogen energy carrier, may be produced with Sodium Hydroxide (NaOH) and the Carbon Monoxide (CO) discharged from the Fuel/Cathode side 308 of the SOEC stack 304.

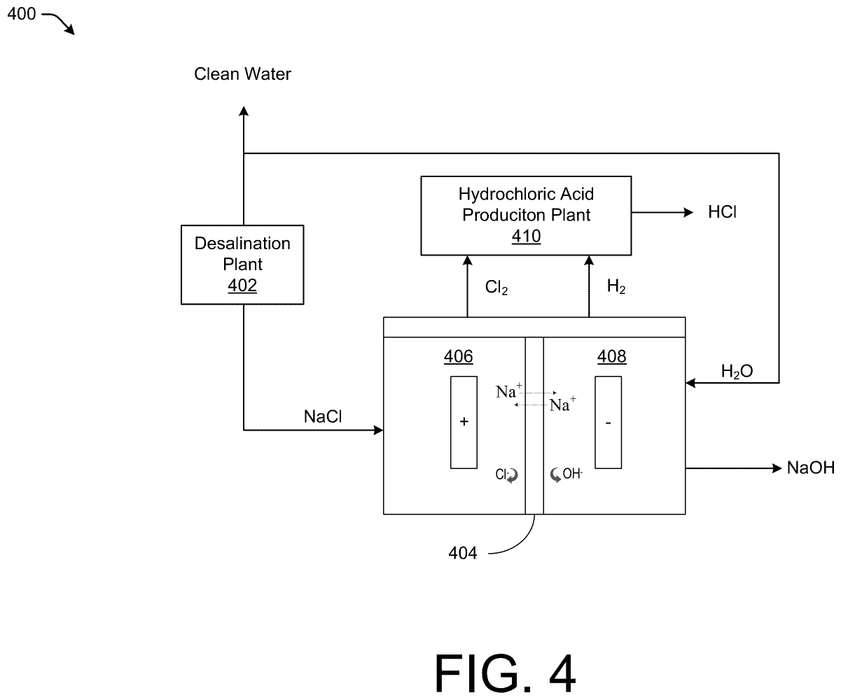

FIG. 4 illustrates a block diagram of a chlor-alkali membrane process 400 (“process 400”), according to an embodiment of this disclosure. In an embodiment, the process 400 may include a desalination plant 402, a membrane cell 404, a first chamber 406, a second chamber 408, and a hydrochloric acid production plant 410.

In an embodiment, the process 400 may use the membrane cell 404 to partition a brine solution in a first chamber 406 from a water (H₂O) solution in the second chamber 408. In an embodiment, saline water is fed to the desalination plant 402 (e.g., desalination plant 104, desalination plant 204). In an embodiment, the desalination plant 402 produces clean water and brine (i.e., a concentrated NaCl solution). For example, in an embodiment, saline water (e.g., seawater with a salt concentration of 3.5% Sodium Chloride (NaCl)) may be fed to the desalination plant 402. The desalination plant 402 may convert the saline water into clean water (i.e., <0.05% Sodium Chloride (NaCl) concentration) and brine (i.e., 7.5% Sodium Chloride (NaCl) concentration).

In an embodiment, brine from the desalination plant 402 is fed to the first chamber 406 of the chlor-alkali membrane cell 404. In an embodiment, a portion of the clean water from the desalination plant 402 may be fed to the second chamber 408 of the chlor-alkali membrane cell 404. A typical chlor-alkali membrane process may process brine concentration up to 26% Sodium Chloride (NaCl). In an embodiment, brine from the desalination plant 402 may include a Sodium Chloride (NaCl) concentration up to 26%. In an embodiment, the process 400 may include one or more membrane cells 404. For example, multiple membrane cells 404 may be configured to operate in series, in parallel, or a combination, thereof.

In an embodiment, the membrane cell 404 may be an ion-selective membrane configured to allow Sodium ions (Na⁺) to flow freely across the membrane between the first chamber 406 and the second chamber 408, while Chloride ions (Cl⁻) and Hydroxide ions (OH) are prevented from migrating across the membrane cell 404. An anode is in the first chamber 406, and a cathode is in the second chamber 408. At the anode, Chloride ions (Cl⁻) from the brine solution are oxidized to form Chlorine (Cl₂) gas. At the cathode, water (H₂O) is reduced to Hydroxide ions (OH⁻) and Hydrogen (H₂) gas, releasing Hydroxide ions (OH⁻) into the solution.

Sodium ions (Na⁺) from the brine solution in the first chamber 406 flow across the membrane toward the cathode in the second chamber 408 and combine with Hydroxide ions (OH⁻) to produce a Sodium Hydroxide (NaOH) solution. The Sodium Hydroxide (NaOH) solution may be removed as a product from the second chamber 408. The equation for the overall reaction for the electrolysis of brine is shown in Equation 3:

2NaCl+2H₂O→Cl₂+H₂+2NaOH (3)

In an embodiment, the process 400 may reduce the Sodium Chloride (NaCl) concentration of brine entering the first chamber 406. The outlet stream from the first chamber 406 may, for example, be reduced to a benign sea water concentrations of Sodium Chloride (NaCl) (i.e., 3.5%). Output from the first chamber 406 may be further processed in a downstream membrane cell 404, fed back to the desalination plant 402 process, or safely released back into the environment, such as into an ocean, sea, or lake comprising the same or higher concentration of Sodium Chloride (NaCl) as the output stream.

In an embodiment, the Chlorine (Cl₂) gas and Hydrogen (H₂) gas generated in the process 400 may be removed as a product to be stored, sold, or used in further resource production, such as in a Hydrochloric Acid (HCl) production plant 410. For example, Chlorine (Cl₂) gas and Hydrogen (H₂) gas can be used to produce hydrogen chloride (HCl) gas for the conversion into Hydrochloric Acid (HCl). The reaction equation is shown in Equation 4:

H₂+Cl₂→2HCl (4)

In an embodiment, the desalination plant 402, the membrane cell 404, and the Hydrochloric Acid production plant 410 may be configured to receive power from a power plant system (e.g., power plant system 102, power plant system 202).

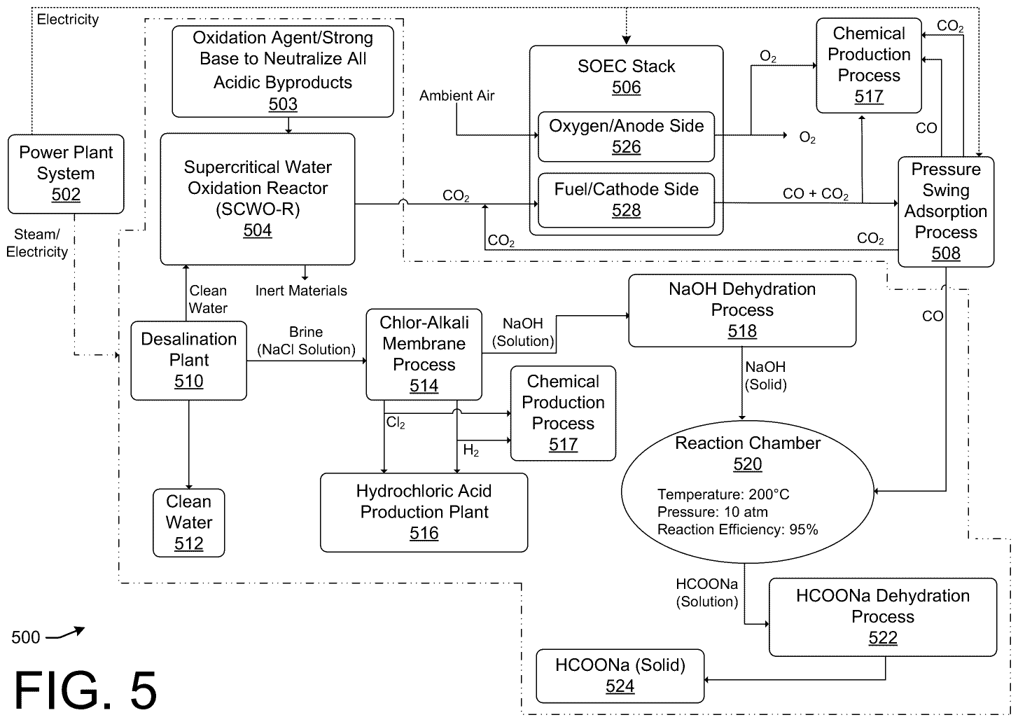

FIG. 5 illustrates a flow diagram of an example system 500 (“system 500”) associated with using a power plant system 502 (e.g., SMR system) and a SCWO-R 504 to produce Sodium Formate (HCOONa) 524, according to an embodiment of this disclosure.

In an embodiment, the system 500 may include the power plant system 502, the oxidation agent/strong base to neutralize all acidic byproducts 503, the supercritical water oxidation reactor (SCWO-R) 504, the SOEC stack 506 (e.g., CO-electrolysis Cell, etc.), the pressure swing adsorption process 508, the desalination plant 510, clean water 512, the chlor-alkali membrane process 514, the Hydrochloric Acid production plant 516, the chemical production process 517, the NaOH dehydration process 518, the reaction chamber 520, the HCOONa dehydration process 522, and the HCOONa (solid) 524.

In an embodiment, the oxidation agent/strong base to neutralize all acidic byproducts 503 may include Oxygen (O₂), Hydrogen Peroxide (H₂O₂), or Sodium Hydroxide (NaOH). In an embodiment, the oxidation agent/strong base to neutralize all acidic byproducts 503 may include Oxygen (O₂) (e.g., oxygen produced by the SOEC stack 506, or any other source of Oxygen (O₂), etc.), Hydrogen Peroxide (H₂O₂), or Sodium Hydroxide (NaOH) (e.g., the Sodium Hydroxide (NaOH) solution produced by the chlor-alkali membrane process 514, or any other NaOH source, etc.). In an embodiment, the SOEC stack 506 may include an Oxygen/Anode side 526 and a Fuel/Cathode side 528.

In an embodiment, the power plant system 502 may be electrically coupled to the to the SCWO-R 504, the SOEC stack 506, the pressure swing adsorption process 508, the desalination plant 510, the chlor-alkali membrane process 514, the Hydrochloric Acid (HCl) production plant 516, the NaOH dehydration process 518, the reaction chamber 520, the HCOONa dehydration process 522, and/or additional components for resource production and/or SCWO operations for selectively providing electricity (e.g., power) thereto.

In an embodiment, individual ones of steam output paths of the power plant system 502 may be fluidly coupled to the SCWO-R 504, the desalination plant 510, the chlor-alkali membrane process 514, the reaction chamber 520, and/or additional components for resource production and/or SCWO operations for selectively providing steam thereto. In an embodiment, the power plant system 502 may be operably coupled to additional or fewer outputs and/or the various outputs can receive electricity and/or steam from other sources (e.g., conventional steam suppliers, conventional electricity sources, etc.).

In an embodiment, the desalination plant 510 may be configured to receive steam from the power plant system 502, electricity from the power plant system 502, and seawater and produce clean water 512 (i.e., water used for industrial processing, hospitals, homes, etc.), brine (i.e., concentrated NaCl solution), and clean water for use in the SCWO-R 504. In an embodiment, the SCWO-R 504 may be configured to receive clean water from the desalination plant 510 to treat biomass and/or organic waste. While treating the biomass and/or organic waste, the SCWO-R 504 may produce Carbon Dioxide (CO₂) that may be directed to the Fuel/Cathode side 528 of the SOEC stack 506.

In an embodiment, the Oxygen/Anode side 526 of the SOEC stack 506 may receive ambient air. The SOEC stack 506 may process the Carbon Dioxide (CO₂) and the ambient air to produce Oxygen (O₂) and a mixture of Carbon Monoxide (CO) and Carbon Dioxide (CO₂). The Carbon Monoxide (CO) and Carbon Dioxide (CO₂) mixture may be directed to the pressure swing adsorption process 508 to be separated. In an embodiment, the Carbon Monoxide (CO) and Carbon Dioxide (CO₂) mixture may be directed to the chemical production process 517 (e.g., Methanol (CH₃OH) production, Acetic Acid (CH₃COOH) production, Formaldehyde (CH₂O) production, Methyl Tertiary-Butyl Ether (MTBE) (C₅H₁₂O) production, Dimethyl Ether (DME) (CH₃OCH₃), etc.). In an embodiment, the Carbon Dioxide (CO₂) separated from the Carbon Monoxide (CO) and Carbon Dioxide (CO₂) mixture may be directed back to the inlet of the Fuel/Cathode side 528 of the SOEC stack 506, while the Carbon Monoxide (CO) separated from the Carbon Monoxide (CO) and Carbon Dioxide (CO₂) mixture may be directed to the reaction chamber 520. In an embodiment, the Carbon Dioxide (CO₂) and the Carbon Monoxide (CO) separated from the Carbon Monoxide (CO) and Carbon Dioxide (CO₂) mixture may be directed to the chemical production process 517 for chemical production (e.g., Methanol (CH₃OH) production, Acetic Acid (CH₃COOH) production, Formaldehyde (CH₂O) production, Methyl Tertiary-Butyl Ether (MTBE) (C₅H₁₂O) production, Dimethyl Ether (DME) (CH₃OCH₃), etc.).

In an embodiment, the chlor-alkali membrane process 514 may receive brine from the desalination plant 510. The chlor-alkali membrane process 514 may convert the brine to a Sodium Hydroxide (NaOH) solution, Chlorine (Cl₂) gas, and Hydrogen (H₂) gas. In an embodiment, the Chlorine (Cl₂) gas and Hydrogen (H₂) gas may be fed into the Hydrochloric Acid production plant 516. In an embodiment, the Chlorine (Cl₂) gas and Hydrogen (H₂) gas may be fed into the chemical production process 517 for chemical production (e.g., Methanol (CH₃OH) production, Acetic Acid (CH₃COOH) production, Formaldehyde (CH₂O) production, Methyl Tertiary-Butyl Ether (MTBE) (C₅H₁₂₀) production, Dimethyl Ether (DME) (CH₃OCH₃), etc.). In an embodiment, the Sodium Hydroxide (NaOH) solution may be directed to the NaOH dehydration process 518.

In an embodiment, the NaOH dehydration process 518 may be configured to receive the NaOH solution from the chlor-alkali membrane process 514 and produce solid Sodium Hydroxide (NaOH). The solid Sodium Hydroxide (NaOH) may be fed into the reaction chamber 520 for additional processing.

In an embodiment, the reaction chamber 520 may be configured to receive the solid Sodium Hydroxide (NaOH) from the NaOH dehydration process 518 and the Carbon Monoxide (CO) from the pressure swing adsorption process 508. In an embodiment, the reaction chamber may receive steam and/or electricity from the power plant system 502 to achieve an internal temperature and internal pressure of approximately 200° C. and 10 atm, respectively. In an embodiment, the reaction chamber 520 may convert the solid Sodium Hydroxide (NaOH) and Carbon Monoxide (CO) to wet Sodium Formate (HCOONa) (i.e., Sodium Formate (HCOONa) solution). Because Sodium Formate (HCOONa) is hygroscopic, the Sodium Formate (HCOONa) produced by the reaction chamber 520 may include moisture. In an embodiment, the Sodium Formate (HCOONa) solution may be directed to the HCOONa dehydration process 522.

In an embodiment, the HCOONa dehydration process 522 may utilize electricity from the power plant system 502 to remove excess moisture from the Sodium Formate (HCOONa) solution to produce solid Sodium Formate (HCOONa) 524. In an embodiment, the solid Sodium Formate (HCOONa) 524 may be stored, transported, and/or used for additional processing (e.g., thermally decomposed to produce Hydrogen (H₂) gas, which is an energy carrier).

In an embodiment, Sodium Formate (HCOONa) may be produced when solid Sodium Hydroxide (NaOH) absorbs Carbon Monoxide (CO) at a moderate temperature and pressure (i.e., between 130° C.-150° C. and ˜10 bar pressure). Presently, it is known that an Electric Thermal Vacuum Chamber (ETVC) may be used to dehydrate an NaOH solution to produce solid NaOH. Therefore, the solid Sodium Hydroxide (NaOH) generated via the chlor-alkali membrane process 514 and dehydrated via the NaOH dehydration process 518 may be used to produce Sodium Formate (HCOONa) 524 by combining the solid NaOH with CO produced by an SOEC stack 506 and separated via the pressure swing adsorption process 508. The Sodium Formate (HCOONa) 524 may be stored and/or transported downstream for additional chemical processing (e.g., thermally decomposed to produce Hydrogen (H₂) gas, which is an energy carrier).

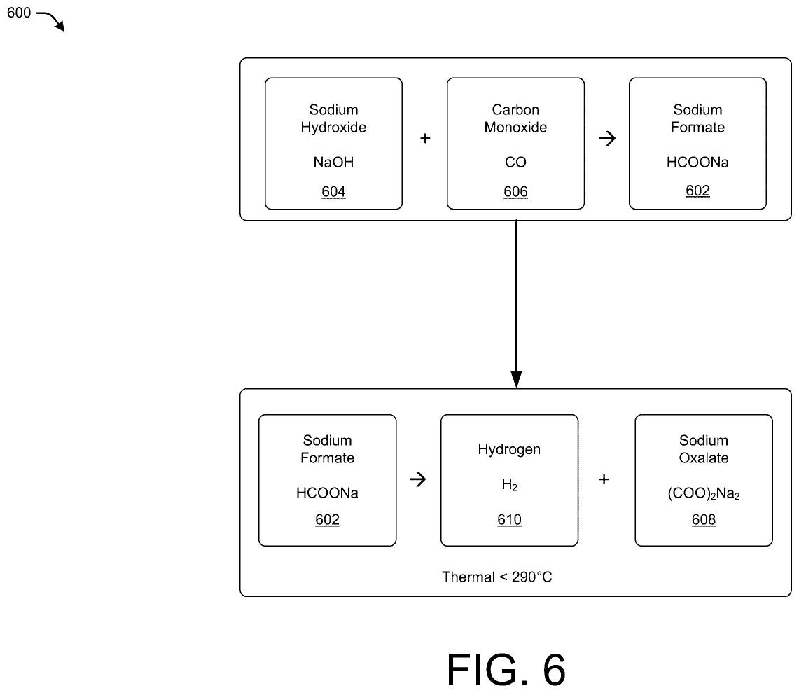

FIG. 6 schematically illustrates an example process 600 (“process 600”) for the generation of Sodium Formate (HCOONa) 602 from Sodium Hydroxide (NaOH) 604 and Carbon Monoxide (CO) 606, and the thermal decomposition of the Sodium Formate (HCOONa) 602 to Sodium Oxalate ((COO)₂Na₂)) 608 and Hydrogen (H₂) 610, according to an embodiment of this disclosure.

FIG. 6 is a simple representation for an alternate production of Sodium Formate (HCOONa) via the reaction between Sodium Hydroxide (NaOH) and Carbon Monoxide (CO) and the subsequent decomposition of Sodium Formate (HCOONa) to generate Hydrogen (H₂) as an energy carrier.

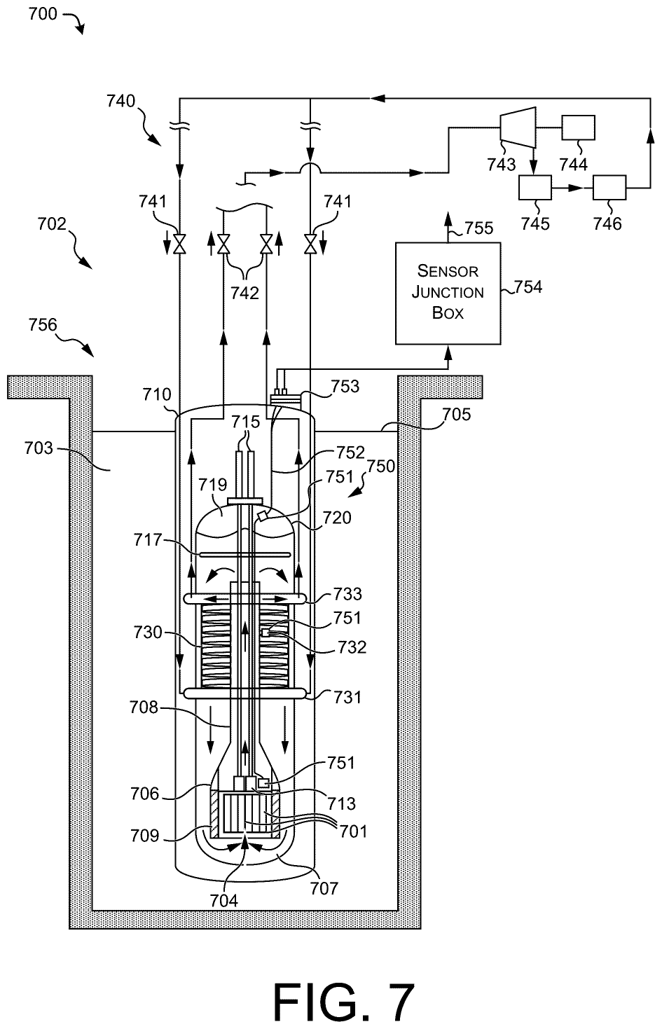

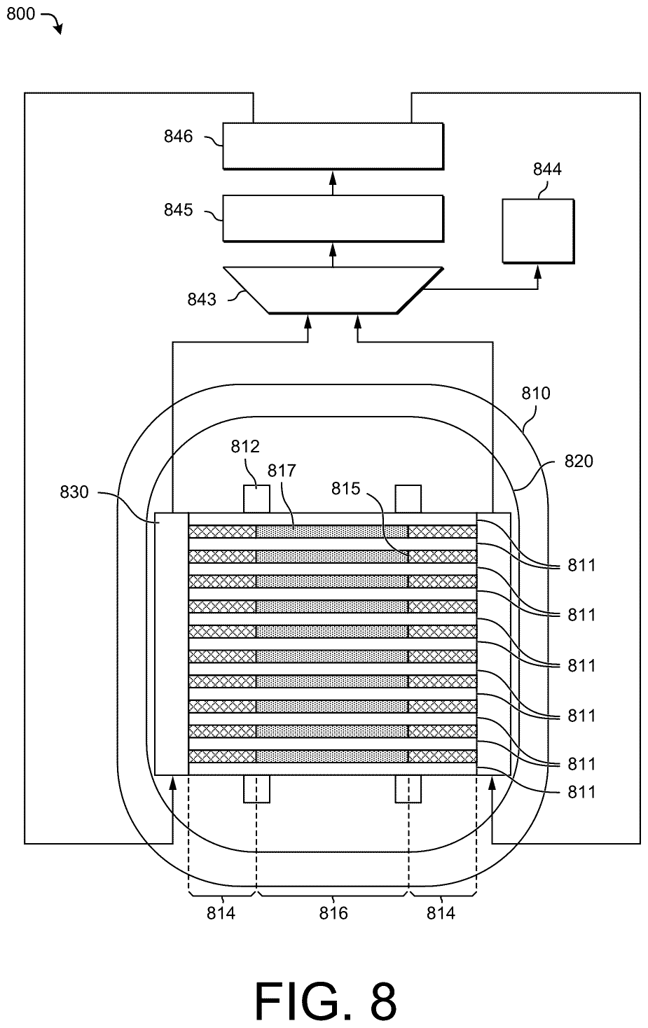

FIGS. 7 and 8 illustrate representative nuclear reactors that may be included in embodiments of the present technology. FIG. 7 is a partially schematic, partially cross-sectional view of a nuclear reactor system 700 configured in accordance with embodiments of the present technology. The system 700 can include a power module 702 having a reactor core 704 in which a controlled nuclear reaction takes place. Accordingly, the reactor core 704 can include one or more fuel assemblies 701. The fuel assemblies 701 can include fissile and/or other suitable materials. Heat from the reaction generates steam at a steam generator 730, which directs the steam to a power conversion system 740. The power conversion system 740 generates electrical power, and/or provides other useful outputs, such as super-heated steam. A sensor system 750 is used to monitor the operation of the power module 702 and/or other system components. The data obtained from the sensor system 750 can be used in real time to control the power module 702, and/or can be used to update the design of the power module 702 and/or other system components.

The power module 702 includes a containment vessel 710 (e.g., a radiation shield vessel, or a radiation shield container) that houses/encloses a reactor vessel 720 (e.g., a reactor pressure vessel, or a reactor pressure container), which in turn houses the reactor core 704. The containment vessel 710 can be housed in a power module bay 756. The power module bay 756 can contain a cooling pool 703 filled with water and/or another suitable cooling liquid. The bulk of the power module 702 can be positioned below a surface 705 of the cooling pool 703. Accordingly, the cooling pool 703 can operate as a thermal sink, for example, in the event of a system malfunction.

A volume between the reactor vessel 720 and the containment vessel 710 can be partially or completely evacuated to reduce heat transfer from the reactor vessel 720 to the surrounding environment (e.g., to the cooling pool 703). However, in other embodiments the volume between the reactor vessel 720 and the containment vessel 710 can be at least partially filled with a gas and/or a liquid that increases heat transfer between the reactor vessel 720 and the containment vessel 710. For example, the volume between the reactor vessel 720 and the containment vessel 710 can be at least partially filled (e.g., flooded with the primary coolant 707) during an emergency operation.

Within the reactor vessel 720, a primary coolant 707 conveys heat from the reactor core 704 to the steam generator 730. For example, as illustrated by arrows located within the reactor vessel 720, the primary coolant 707 is heated at the reactor core 704 toward the bottom of the reactor vessel 720. The heated primary coolant 707 (e.g., water with or without additives) rises from the reactor core 704 through a core shroud 706 and to a riser tube 708. The hot, buoyant primary coolant 707 continues to rise through the riser tube 708, then exits the riser tube 708 and passes downwardly through the steam generator 730. The steam generator 730 includes a multitude of conduits 732 that are arranged circumferentially around the riser tube 708, for example, in a helical pattern, as is shown schematically in FIG. 7. The descending primary coolant 707 transfers heat to a secondary coolant (e.g., water) within the conduits 732, and descends to the bottom of the reactor vessel 720 where the cycle begins again. The cycle can be driven by the changes in the buoyancy of the primary coolant 707, thus reducing or eliminating the need for pumps to move the primary coolant 707.

The steam generator 730 can include a feedwater header 731 at which the incoming secondary coolant enters the steam generator conduits 732. The secondary coolant rises through the conduits 732, converts to vapor (e.g., steam), and is collected at a steam header 733. The steam exits the steam header 733 and is directed to the power conversion system 740.

The power conversion system 740 can include one or more steam valves 742 that regulate the passage of high pressure, high temperature steam from the steam generator 730 to a steam turbine 743. The steam turbine 743 converts the thermal energy of the steam to electricity via a generator 744. The low-pressure steam exiting the turbine 743 is condensed at a condenser 745, and then directed (e.g., via a pump 746) to one or more feedwater valves 741. The feedwater valves 741 control the rate at which the feedwater re-enters the steam generator 730 via the feedwater header 731. In other embodiments, the steam from the steam generator 730 can be routed for direct use in an industrial process, such as a Hydrogen (H₂) and Oxygen (O₂) production plant, a chemical production plant, and/or the like, as described in detail below. Accordingly, steam exiting the steam generator 730 can bypass the power conversion system 740.

The power module 702 includes multiple control systems and associated sensors. For example, the power module 702 can include a hollow cylindrical reflector 709 that directs neutrons back into the reactor core 704 to further the nuclear reaction taking place therein. Control rods 713 are used to modulate the nuclear reaction and are driven via fuel rod drivers 715. The pressure within the reactor vessel 720 can be controlled via a pressurizer plate 717 (which can also serve to direct the primary coolant 707 downwardly through the steam generator 730) by controlling the pressure in a pressurizing volume 719 positioned above the pressurizer plate 717.

The sensor system 750 can include one or more sensors 751 positioned at a variety of locations within the power module 702 and/or elsewhere, for example, to identify operating parameter values and/or changes in parameter values. The data collected by the sensor system 750 can then be used to control the operation of the system 700, and/or to generate design changes for the system 700. For sensors positioned within the containment vessel 710, a sensor link 752 directs data from the sensors to a flange 753 (at which the sensor link 752 exits the containment vessel 710) and directs data to a sensor junction box 754. From there, the sensor data can be routed to one or more controllers and/or other data systems via a data bus 755.

FIG. 8 is a partially schematic, partially cross-sectional view of a nuclear reactor system 800 configured in accordance with additional embodiments of the present technology. In some embodiments, the nuclear reactor system 800 (“system 800”) can include some features that are at least generally similar in structure and function, or identical in structure and function, to the corresponding features of the system 800 described in detail above with reference to FIG. 8 and can operate in a generally similar or identical manner to the system 800.

In the illustrated embodiment, the system 800 includes a reactor vessel 820 and a containment vessel 810 surrounding/enclosing the reactor vessel 820. In some embodiments, the reactor vessel 820 and the containment vessel 810 can be roughly cylinder-shaped or capsule-shaped. The system 800 further includes a plurality of heat pipe layers 811 within the reactor vessel 820. In the illustrated embodiment, the heat pipe layers 811 are spaced apart from and stacked over one another. In some embodiments, the heat pipe layers 811 can be mounted/secured to a common frame 812, a portion of the reactor vessel 820 (e.g., a wall thereof), and/or other suitable structures within the reactor vessel 820. In other embodiments, the heat pipe layers 811 can be directly stacked on top of one another such that each of the heat pipe layers 811 supports and/or is supported by one or more of the other ones of the heat pipe layers 811.

In the illustrated embodiment, the system 800 further includes a shield or reflector region 814 at least partially surrounding a core region 816. The heat pipe layers 811 can be circular, rectilinear, polygonal, and/or can have other shapes, such that the core region 816 has a corresponding three-dimensional shape (e.g., cylindrical, spherical). In some embodiments, the core region 816 is separated from the reflector region 814 by a core barrier 815, such as a metal wall. The core region 816 can include one or more fuel sources, such as fissile material, for heating the heat pipe layers 811. The reflector region 814 can include one or more materials configured to contain/reflect products generated by burning the fuel in the core region 816 during operation of the system 800. For example, the reflector region 814 can include a liquid or solid material configured to reflect neutrons and/or other fission products radially inward toward the core region 816. In some embodiments, the reflector region 814 can entirely surround the core region 816. In other embodiments, the reflector region 814 may partially surround the core region 816. In some embodiments, the core region 816 can include a control material 817, such as a moderator and/or coolant. The control material 817 can at least partially surround the heat pipe layers 811 in the core region 816 and can transfer heat therebetween.

In the illustrated embodiment, the system 800 further includes at least one heat exchanger 830 (e.g., a steam generator) positioned around the heat pipe layers 811. The heat pipe layers 811 can extend from the core region 816 and at least partially into the reflector region 814 and are thermally coupled to the heat exchanger 830. In some embodiments, the heat exchanger 830 can be positioned outside of or partially within the reflector region 814. The heat pipe layers 811 provide a heat transfer path from the core region 816 to the heat exchanger 830. For example, the heat pipe layers 811 can each include an array of heat pipes that provide a heat transfer path from the core region 816 to the heat exchanger 830. When the system 800 operates, the fuel in the core region 816 can heat and vaporize a fluid within the heat pipes in the heat pipe layers 811, and the fluid can carry the heat to the heat exchanger 830. The heat pipes in the heat pipe layers 811 can then return the fluid toward the core region 816 via wicking, gravity, and/or other means to be heated and vaporized once again.

In some embodiments, the heat exchanger 830 can be similar to the steam generator 730 of FIG. 7 and, for example, can include one or more helically-coiled tubes that wrap around the heat pipe layers 811. The tubes of the heat exchanger 830 can include or carry a working fluid (e.g., a coolant such as water or another fluid) that carries the heat from the heat pipe layers 811 out of the reactor vessel 820 and the containment vessel 810 for use in generating electricity, steam, and/or the like. For example, in the illustrated embodiment the heat exchanger 830 is operably coupled to a turbine 843, a generator 844, a condenser 845, and a pump 846. As the working fluid within the heat exchanger 830 increases in temperature, the working fluid may begin to boil and vaporize. The vaporized working fluid (e.g., steam) may be used to drive the turbine 843 to convert the thermal potential energy of the working fluid into electrical energy via the generator 844. The condenser 845 can condense the working fluid after it passes through the turbine 843, and the pump 846 can direct the working fluid back to the heat exchanger 830 where it can begin another thermal cycle. In other embodiments, steam from the heat exchanger 830 can be routed for direct use in an industrial process, such as an enhanced oil recovery operation described in detail below. Accordingly, steam exiting the heat exchanger 830 can bypass the turbine 843, the generator 844, the condenser 845, the pump 846, etc.

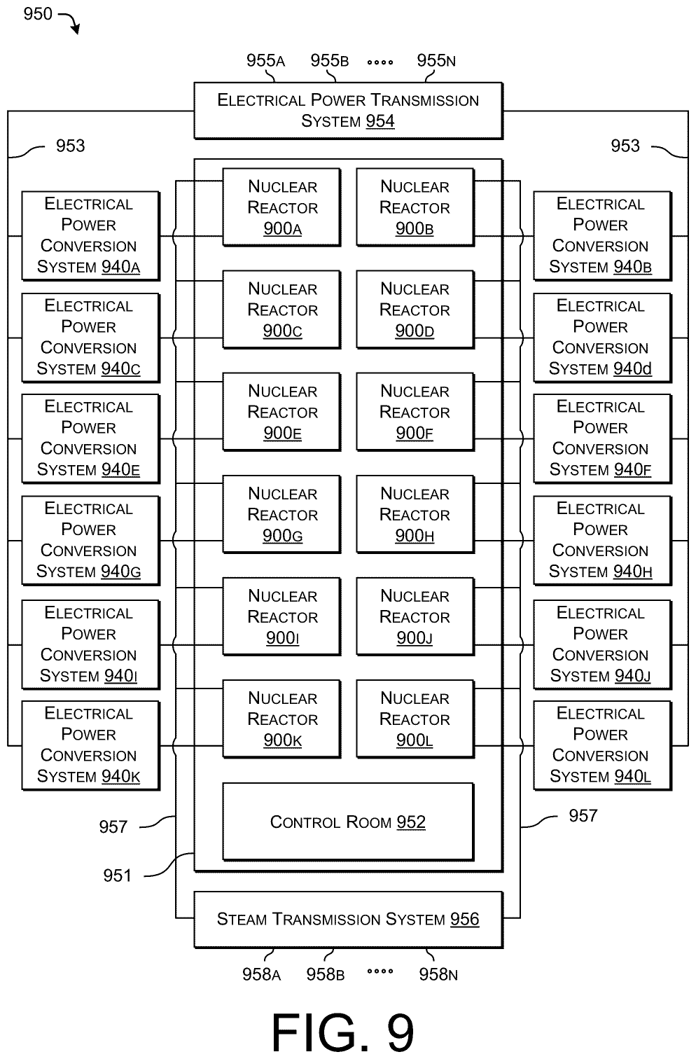

FIG. 9 is a schematic view of a nuclear power plant system 950 including multiple nuclear reactors 900 in accordance with embodiments of the present technology. Each of the nuclear reactors 900 (individually identified as first through twelfth nuclear reactors 900a-1, respectively) can be similar to or identical to the nuclear reactor 900 and/or the nuclear reactor 900 described in detail above with reference to FIGS. 7 and 8. The power plant system 950 (“power plant system 950”) can be “modular” in that each of the nuclear reactors 900 can be operated separately to provide an output, such as electricity or steam. The power plant system 950 can include fewer than twelve of the nuclear reactors 900 (e.g., two, three, four, five, six, seven, eight, nine, ten, or eleven of the nuclear reactors 900), or more than twelve of the nuclear reactors 900. The power plant system 950 can be a permanent installation or can be mobile (e.g., mounted on a truck, tractor, mobile platform, and/or the like). In the illustrated embodiment, each of the nuclear reactors 900 can be positioned within a common housing 951, such as a reactor plant building, and controlled and/or monitored via a control room 952.

Each of the nuclear reactors 900 can be coupled to a corresponding electrical power conversion system 940 (individually identified as first through twelfth electrical power conversion systems 940a-1, respectively). The electrical power conversion systems 940 can include one or more devices that generate electrical power or some other form of usable power from steam generated by the nuclear reactors 900. In some embodiments, multiple ones of the nuclear reactors 900 can be coupled to the same one of the electrical power conversion systems 940 and/or one or more of the nuclear reactors 900 can be coupled to multiple ones of the electrical power conversion systems 940 such that there is not a one-to-one correspondence between the nuclear reactors 900 and the electrical power conversion systems 940.

The electrical power conversion systems 940 can be further coupled to an electrical power transmission system 954 via, for example, an electrical power bus 953. The electrical power transmission system 954 and/or the electrical power bus 953 can include one or more transmission lines, transformers, and/or the like for regulating the current, voltage, and/or other characteristic(s) of the electricity generated by the electrical power conversion systems 940. The electrical power transmission system 454 can route electricity via a plurality of electrical output paths 955 (individually identified as electrical output paths 955a-n) to one or more end users and/or end uses, such as different electrical loads of an integrated energy system.

Each of the nuclear reactors 900 can further be coupled to a steam transmission system 956 via, for example, a steam bus 957. The steam bus 957 can route steam generated from the nuclear reactors 900 to the steam transmission system 956 which in turn can route the steam via a plurality of steam output paths 958 (individually identified as steam output paths 958a-n) to one or more end users and/or end uses, such as different steam inputs of an integrated energy system.

In some embodiments, the nuclear reactors 900 can be individually controlled (e.g., via the control room 952) to provide steam to the steam transmission system 956 and/or steam to the corresponding one of the electrical power conversion systems 940 to provide electricity to the electrical power transmission system 954. In some embodiments, the nuclear reactors 900 are configured to provide steam either to the steam bus 957 or to the corresponding one of the electrical power conversion systems 940 and can be rapidly and efficiently switched between providing steam to either. Accordingly, in some aspects of the present technology the nuclear reactors 900 can be modularly and flexibly controlled such that the power plant system 950 can provide differing levels/amounts of electricity via the electrical power transmission system 954 and/or steam via the steam transmission system 956. For example, where the power plant system 950 is used to provide electricity and steam to one or more industrial process-such as various components of the integrated energy systems, the nuclear reactors 900 can be controlled to meet the differing electricity and steam requirements of the industrial processes.

As one example, during a first operational state of an integrated energy system employing the power plant system 950, a first subset of the nuclear reactors 900 (e.g., the first through sixth nuclear reactors 900a-f) can be configured to provide steam to the steam transmission system 956 for use in the first operational state of the integrated energy system, while a second subset of the nuclear reactors 900 (e.g., the seventh through twelfth nuclear reactors 900g-l) can be configured to provide steam to the corresponding ones of the electrical power conversion systems 940 (e.g., the seventh through twelfth electrical power conversion systems 940g-l) to generate electricity for the first operational state of the integrated energy system. Then, during a second operational state of the integrated energy system when a different (e.g., greater or lesser) amount of steam and/or electricity is required, some or all the first subset of the nuclear reactors 900 can be switched to provide steam to the corresponding ones of the electrical power conversion systems 940 (e.g., the seventh through twelfth electrical power conversion systems 940g-l) and/or some or all of the second subset of the nuclear reactors 900 can be switched to provide steam to the steam transmission system 956 to vary the amount of steam and electricity produced to match the requirements/demands of the second operational state. Other variations of steam and electricity generation are possible based on the needs of the integrated energy system. That is, the nuclear reactors 900 can be dynamically/flexibly controlled during other operational states of an integrated energy system to meet the steam and electricity requirements of the operational state.

In contrast, some conventional nuclear power plant systems can typically generate either steam or electricity for output and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 900 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured, or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

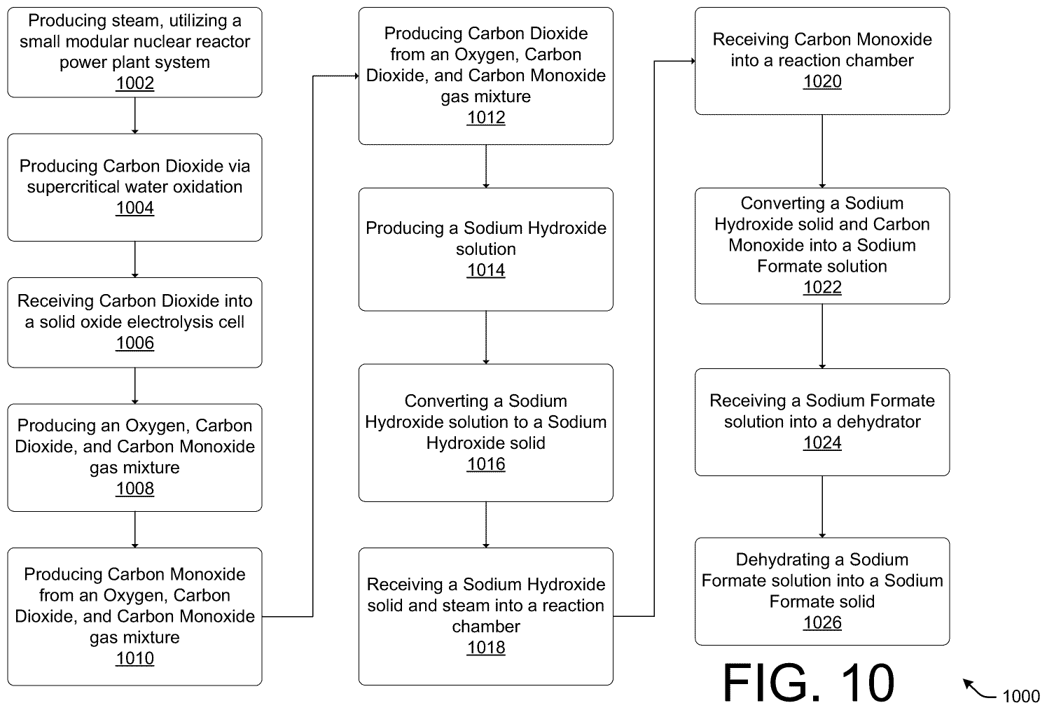

FIG. 10 illustrates a flow diagram of an example process 1000 for utilizing a small modular nuclear reactor (SMR) system to produce Sodium Formate (HCOONa). The order in which the operations or steps are described is not intended to be construed as a limitation, and any number of the described operations may be combined in any order and/or in parallel to implement integrated SMR system (e.g., the production system 500, as discussed above with reference to FIG. 5).

At step 1002, the process 1000 may include producing steam and electricity utilizing an SMR system, as needed for the SMR system. For example, the desalination plant 510, the chlor-alkali membrane process 514, the Hydrochloric Acid (HCl) production plant 516, the chemical production process 517, and the SCWO-R 504, may utilize electricity generated by power plant system 502, as discussed above regarding FIG. 5.

At step 1004, the process 1000 may include producing Carbon Dioxide (CO₂) via supercritical water oxidation. For example, the SCWO-R 504 of FIG. 5 may utilize steam and electricity from the power plant system 502 and clean water from the desalination plant 510 to process biomass and/or organic waste and produce Carbon Dioxide (CO₂), as discussed above regarding FIG. 5.

At step 1006, the process 1000 may include receiving Carbon Dioxide into a solid oxide electrolysis cell. For example, the SOEC stack 506 (e.g., CO Electrolysis Cell, etc.) of FIG. 5 may receive Carbon Dioxide (CO₂) from the SCWO-R 504 and air, as discussed above regarding FIG. 5.

At step 1008, the process 1000 may include producing an Oxygen, Carbon Dioxide, and Carbon Monoxide gas mixture. For example, the SOEC stack 506 (e.g., CO Electrolysis Cell) of FIG. 5 may include the Oxygen/Anode side 526 that may receive the ambient air and the Fuel/Cathode side 528 that may receive the Carbon Dioxide (CO₂) from the SCWO-R 504. The SOEC stack 506 may convert the ambient air and the Carbon Dioxide (CO₂) from the SCWO-R 504 into Oxygen (O₂) gas and a mixture of Carbon Monoxide (CO) and Carbon Dioxide (CO₂), as discussed above regarding FIG. 5. At step 1010, the process 1000 may include producing Carbon Monoxide from an Oxygen, Carbon Dioxide, and Carbon Monoxide gas mixture. For example, the pressure swing adsorption process 508 of FIG. 5 may receive a mixture of Oxygen (O₂), Carbon Monoxide (CO), and Carbon Dioxide (CO₂) from the SOEC stack 506. The pressure swing adsorption process 508 may separate the mixture of Carbon Monoxide (CO) and Carbon Dioxide (CO₂) to separate Carbon Monoxide (CO) and Carbon Dioxide (CO₂) gases, as discussed above regarding FIG. 5.

At step 1012, the process 1000 may include producing Carbon Dioxide from an Oxygen, Carbon Dioxide, and Carbon Monoxide gas mixture. For example, the pressure swing adsorption process 508 of FIG. 5 may receive a mixture of Oxygen (O₂), Carbon Monoxide (CO), and Carbon Dioxide (CO₂) from the SOEC stack 506. The pressure swing adsorption process 508 may separate the mixture of Carbon Monoxide (CO) and Carbon Dioxide (CO₂) to separate Carbon Monoxide (CO) and Carbon Dioxide (CO₂) gases, as discussed above regarding FIG. 5.

At step 1014, the process 1000 may include producing a Sodium Hydroxide solution. For example, the chlor-alkali membrane process 514 of FIG. 5 may receive brine (i.e., a concentrated NaCl solution) from the desalination plant 510. The chlor-alkali membrane process 514 may be convert the brine to a Sodium Hydroxide (NaOH) solution, Chlorine (Cl₂) gas, and Hydrogen (H₂) gas, as discussed above regarding FIG. 5.

At step 1016, the process 1000 may include converting a Sodium Hydroxide solution to a Sodium Hydroxide solid. For example, the NaOH dehydration process 508 of FIG. 5 may receive the Sodium Hydroxide (NaOH) solution from the chlor-alkali membrane process 514 and remove moisture to produce a Sodium Hydroxide (NaOH) solid, as discussed above regarding FIG. 5.

At step 1018, the process 1000 may include receiving a Sodium Hydroxide solid and steam into a reaction chamber. For example, the reaction chamber 520 of FIG. 5 may receive the Sodium Hydroxide (NaOH) solid from the NaOH dehydration process 518, as discussed above regarding FIG. 5.

At step 1020, the process 1000 may include receiving Carbon Monoxide into a reaction chamber. For example, the reaction chamber 520 of FIG. 5 may receive Carbon Monoxide (CO) from the pressure swing adsorption process 508, as discussed above regarding FIG. 5.

At step 1022, the process 1000 may include converting a Sodium Hydroxide solid and Carbon Monoxide into a Sodium Formate solution. For example, the reaction chamber 520 of FIG. 5 may receive a Sodium Hydroxide solid from the dehydration process 518. The reaction chamber 520 may receive steam and/or electricity from the power plant system 502 to maintain the reaction chamber at approximately 200° C. and 10 atm. to facilitate a reaction between the Sodium Hydroxide (NaOH) solid and the Carbon Monoxide (CO) to produce a Sodium Formate (HCOONa) solution, as discussed above regarding FIG. 5.

At step 1024, the process 1000 may include receiving a Sodium Formate solution into a dehydrator. For example, the HCOONa dehydration process 522 of FIG. 5 may receive a Sodium Formate (HCOONa) solution from the reaction chamber 520, as discussed above regarding FIG. 5.

At step 1026, the process 1000 may include dehydrating a Sodium Formate solution into a Sodium Formate solid. For example, the HCOONa dehydration process 522 of FIG. 5 may remove moisture from the Sodium Formate (HCOONa) solution to produce the Sodium Formate (HCOONa) solid 524, as discussed above regarding FIG. 5.

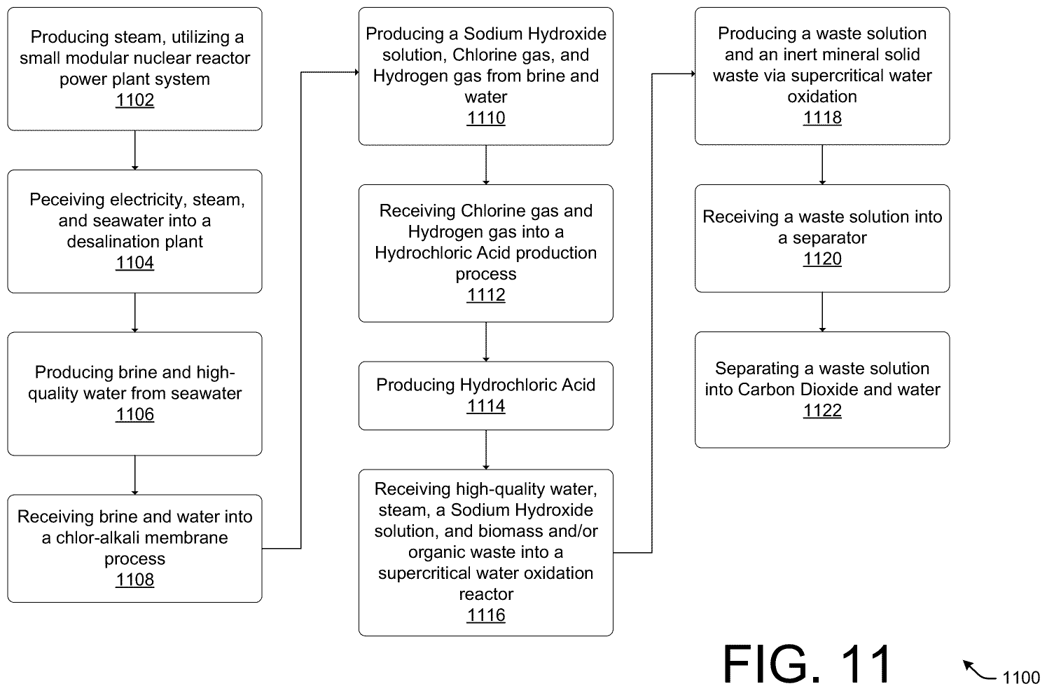

FIG. 11 illustrates a flow diagram of an example process 1100 for utilizing a small modular nuclear reactor (SMR) system and supercritical water oxidation to treat biomass and/or organic waste. The order in which the operations or steps are described is not intended to be construed as a limitation, and any number of the described operations may be combined in any order and/or in parallel to implement integrated SMR system (e.g., the production system 200, as discussed above with reference to FIG. 2).

At step 1102, the process 1100 may include producing steam and electricity utilizing an SMR system, as needed for the SMR system. For example, the desalination plant 204, the chlor-alkali membrane process 212, the Hydrochloric Acid production process 216, the SCWO-R 224, and the separator 230 may utilize electricity generated by power plant system 202, as discussed above regarding FIG. 2.

At step 1104, the process 1100 may include receiving electricity, steam, and seawater into a desalination plant. For example, the desalination plant 204 may of FIG. 2 may receive electricity and steam from the power plant system 202 and seawater, as discussed above regarding FIG. 2.

At step 1106, the process 1100 may include producing brine and high-quality water from seawater. For example, the desalination plant 204 of FIG. 2 may receive seawater and produce brine and high-quality water, as discussed above regarding FIG. 2.

At step 1108, the process 1100 may include receiving brine and water into a chlor-alkali membrane process. For example, the chlor-alkali membrane process 212 may receive brine from the desalination plant 204 and water from the water source 214 (which, in an embodiment, may also be the desalination plant 204), as discussed above regarding FIG. 2.

At step 1110, the process 1100 may include producing a Sodium Hydroxide (NaOH) solution, Chlorine (Cl₂) gas, and Hydrogen (H₂) gas from brine and water. For example, the chlor-alkali membrane process 212 may produce at least one Sodium Hydroxide (NaOH) solution, Chlorine (Cl₂) gas, and Hydrogen (H₂) gas, as discussed above regarding FIG. 2.

At step 1112, the process 1100 may include receiving Chlorine (Cl₂) gas and Hydrogen (O₂) gas into a Hydrochloric Acid production process. For example, the Hydrochloric Acid production process 216 may receive the Chlorine (Cl₂) gas and Hydrogen (H₂) gas produced by the chlor-alkali membrane process 212, as discussed above regarding FIG. 2.

At step 1114, the process 1100 may include producing Hydrochloric Acid. For example, the Hydrochloric Acid production process 216 may receive the Chlorine (Cl₂) gas and Hydrogen (H₂) gas produced by the chlor-alkali membrane process 212 and produce Hydrochloric Acid (HCl), as discussed above regarding FIG. 2.

At step 1116, the process 1100 may include receiving high-quality water, steam, a Sodium Hydroxide (NaOH) solution, and biomass and/or organic waste into a supercritical water oxidation reactor. For example, the SCWO-R 224 may receive steam from the power plant system 202, high-quality water produced by the desalination plant 204, an NaOH solution produced by the chlor-alkali membrane process 212, and biomass and/or organic waste 226, as discussed above regarding FIG. 2.

At step 1118, the process 1100 may include producing a waste solution and an inert mineral solid waste via supercritical water oxidation. For example, the SCWO-R 224 may receive steam from the power plant system 202, high-quality water produced by the desalination plant 204, an NaOH solution produced by the chlor-alkali membrane process 212, and biomass and/or organic waste 226 to produce a waste solution and an inert mineral solid waste 228, as discussed above regarding FIG. 2.

At step 1120, the process 1100 may include receiving a waste solution into a separator. For example, the separator 230 may receive the waste solution and the inert mineral solid waste 228 produced in the SCWO-R 224 via the supercritical water oxidation of the biomass and/or organic waste 226, as discussed above regarding FIG. 2.

At step 1122, the process 1100 may include separating a waste solution into Carbon Dioxide (CO₂) and water. For example, the separator 230 may receive the waste solution produced by the SCWO-R 224 via the supercritical water oxidation of the biomass and/or organic waste 226 and separate the waste solution into the Carbon Dioxide (CO₂) 232 and the water 234, as discussed above regarding FIG. 2.

### CONCLUSION

Although several embodiments have been described in language specific to structural features and/or methodological acts, it is to be understood that the claims are not necessarily limited to the specific features or acts described. Rather, the specific features and acts are disclosed as illustrative forms of implementing the claimed subject matter.

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. An integrated energy system comprising:
a power plant including at least one nuclear reactor and an electrical power generation system, the at least one nuclear reactor being configured to generate steam; and
a supercritical water oxidation system operably coupled to the power plant, the supercritical water oxidation system including:
a desalination plant configured to produce first water and brine,
a chlor-alkali membrane process configured to receive the brine and produce at least a Sodium Hydroxide solution,
a reactor configured to receive the first water, the steam, and the Sodium Hydroxide solution to produce a waste solution and a solid waste, and
a separator configured to receive the waste solution and produce Carbon Dioxide and second water.

2. The integrated energy system of claim 1, wherein the supercritical water oxidation system further includes a Hydrochloric Acid production plant configured to combine Chlorine gas and Hydrogen gas from the chlor-alkali membrane process to generate Hydrochloric Acid.

3. The integrated energy system of claim 1, the supercritical water oxidation system further including a compressor and heater configured to receive the first water at a first temperature and first pressure and produce the first water at a second temperature and a second pressure, wherein the second temperature is greater than the first temperature and the second pressure is greater than the first pressure.

4. The integrated energy system of claim 1, the supercritical water oxidation system further including a pre-heater configured to receive the Sodium Hydroxide solution at a first temperature and produce the Sodium Hydroxide solution at a second temperature, wherein the second temperature is greater than the first temperature.

5. A method for Sodium Formate production, comprising:
producing steam, utilizing a small modular nuclear reactor power plant system;
producing first Carbon Dioxide via supercritical water oxidation using a supercritical water oxidation reactor;
receiving the first Carbon Dioxide into a solid oxide electrolysis cell;
producing a Carbon Dioxide and Carbon Monoxide gas mixture via the solid oxide electrolysis cell;
producing Carbon Monoxide from the Carbon Dioxide and Carbon Monoxide gas mixture via a pressure swing adsorption process;
producing second Carbon Dioxide from the Carbon Dioxide and Carbon Monoxide gas mixture via a pressure swing adsorption process;
producing a Sodium Hydroxide solution via a chlor-alkali membrane process;
converting the Sodium Hydroxide solution, via a Sodium Hydroxide dehydration process, to a Sodium Hydroxide solid;
receiving the Sodium Hydroxide solid into a reaction chamber, the reaction chamber receiving a portion of the steam;
receiving the Carbon Monoxide into the reaction chamber;
converting the Sodium Hydroxide solid and the Carbon Monoxide into a Sodium Formate solution;
receiving the Sodium Formate solution into a dehydrator; and
dehydrating the Sodium Formate solution into a Sodium Formate solid.

6. The method of claim 5, further comprising producing Chlorine gas and Hydrogen gas via the chlor-alkali membrane process.

7. The method of claim 6, further comprising:
receiving the Chlorine gas and the Hydrogen gas into a Hydrochloric Acid production plant; and
producing, via the Hydrochloric Acid production plant, Hydrochloric Acid using the Chlorine gas and the Hydrogen gas.

8. The method of claim 6, further comprising:
receiving the Hydrogen gas into a Methanol production plant;
receiving the Carbon Monoxide into a Methanol production plant;
receiving the second Carbon Dioxide into a Methanol production plant; and
producing, via the Methanol production plant, Methanol using the Hydrogen gas, the Carbon Monoxide, and the second Carbon Dioxide.

9. The method of claim 6, further comprising:
receiving the Hydrogen gas into a Formaldehyde production plant;
receiving the Carbon Monoxide into a Formaldehyde production plant;
receiving the second Carbon Dioxide into a Formaldehyde production plant; and
producing, via the Formaldehyde production plant, Formaldehyde using the Hydrogen gas, the Carbon Monoxide, and the second Carbon Dioxide.

10. The method of claim 5, wherein the reaction chamber has a temperature of approximately 200° C. and a pressure of approximately 10 atm.

11. The method of claim 5, wherein the supercritical water oxidation reactor may be configured to continuously receive the steam to maintain a temperature greater than 375° C.

12. The method of claim 5, wherein producing the first Carbon Dioxide via the supercritical water oxidation using the supercritical water oxidation reactor includes:
producing, using the supercritical water oxidation reactor, a waste solution,
receiving the waste solution into a separator, and
producing, via the separator, the second Carbon Dioxide and water.

13. A system for Sodium Formate production, comprising:
a small modular nuclear reactor (SMR) power plant system configured to supply steam;
a supercritical water oxidation reactor configured to produce first Carbon Dioxide via supercritical water oxidation;
a solid oxide electrolysis cell configured to receive the first Carbon Dioxide to produce a Carbon Dioxide and Carbon Monoxide gas mixture;
a pressure swing adsorption process configured to produce Carbon Monoxide and second Carbon Dioxide from the Carbon Dioxide and Carbon Monoxide gas mixture;
a chlor-alkali membrane process configured to produce a Sodium Hydroxide solution;
a Sodium Hydroxide dehydration process configured to convert the Sodium Hydroxide solution to a Sodium Hydroxide solid;
a reaction chamber configured to:
receive a portion of the steam,
receive the Sodium Hydroxide solid,
receive the Carbon Monoxide, and
convert the Sodium Hydroxide solid and the Carbon Monoxide to a Sodium Formate solution; and
a dehydrator configured to receive the Sodium Formate solution and dehydrate the Sodium Formate solution into a Sodium Formate solid.

14. The system of claim 13, the chlor-alkali membrane process further configured to produce Chlorine gas and Hydrogen gas.

15. The system of claim 14, further comprising a Hydrochloric Acid production plant configured to:
receive the Chlorine gas and the Hydrogen gas, and
produce Hydrochloric Acid using the Chlorine gas and the Hydrogen gas.

16. The system of claim 14, wherein producing the first Carbon Dioxide via the supercritical water oxidation using the supercritical water oxidation reactor includes:
producing, via the supercritical water oxidation reactor, a waste solution,
receiving the waste solution into a separator, and
producing, via the separator, second Carbon Dioxide and water.

17. The system of claim 14, further comprising a Methanol production plant configured to:
receive the Hydrogen gas;
receive the Carbon Monoxide;
receive the second Carbon Dioxide; and
produce Methanol using the Hydrogen gas, the Carbon Monoxide, and the second Carbon Dioxide.

18. The system of claim 14, further comprising a Formaldehyde production plant configured to:
receive the Hydrogen gas;
receive the Carbon Monoxide;
receive the second Carbon Dioxide; and
produce Methanol using the Hydrogen gas, the Carbon Monoxide, and the second Carbon Dioxide.

19. The system of claim 13, the reaction chamber further configured to sustain a temperature of approximately >350° C.

20. The system of claim 13, the supercritical water oxidation reactor further configured to continuously receive the steam to maintain a temperature greater than 375° C. and a pressure greater than a 22.1 MPa.
